(19) United States
(12) Patent Application Publication
(10) Pub. No.: US 2010/0305311 A1
(43) Pub. Date: Dec. 2, 2010

(54) NANOPARTICLE FOR SEPARATING PEPTIDE, METHOD FOR PREPARING THE SAME, AND METHOD FOR SEPARATING PEPTIDE USING THE SAME,

(75) Inventors: Sang-Won Lee, Seoul (KR); Kwang-Yeol Lee, Gyeonggi-do (KR); Je-Sun Lee, Gyeonggi-do (KR); Ji-Young Huh, Seoul (KR); Min-Sik Kim, Seoul (KR); Yeon-Ji Lee, Gyeonggi-do (KR)

Correspondence Address: ALSTON & BIRD LLP, BANK OF AMERICA PLAZA, 101 SOUTH TRYON STREET, SUITE 4000, CHARLOTTE, NC, 28280-4000, US

(73) Assignee: Korea University Industrial & Academic Collaboration Foundation

(21) Appl. No.: 12/745,203

(22) PCT Filed: Nov. 28, 2008
(86) PCT No.: PCT/KR08/07033

§ 371 Date: Aug. 18, 2010

(30) Foreign Application Priority Data
Nov. 30, 2007 (KR) 10-2007-0123943
Nov. 27, 2008 (KR) 10-2008-0119056

(51) Int. Cl. C07K 1/36; C07F 15/02; H01F 1/01; B05D 5/00; B32B 5/16

(52) U.S. Cl. 530/415; 546/2; 252/62.51 R; 252/62.56; 427/127; 428/402

(57) ABSTRACT

Disclosed are nanoparticles for use in the isolation of peptides, a method for producing the nanoparticles, and a method for the isolation of peptides using the nanoparticles. The nanoparticles comprise magnetic nanoparticles and thiol-specific functional groups as first functional groups bound to the surfaces of the magnetic nanoparticles to selectively capture cysteine-containing peptides. The nanoparticles allow highly selective isolation of target peptides in a simple and rapid manner. Therefore, the nanoparticles can be applied to research on the treatment of diseases such as cancers.

### TECHNICAL FIELD

The present invention relates to nanoparticles for use in the isolation of peptides, a method for producing the nanoparticles, and a method for the isolation of peptides using the nanoparticles. More specifically, the nanoparticles comprise magnetic nanoparticles and thiol-specific functional groups as first functional groups bound to the surfaces of the magnetic nanoparticles to selectively capture cysteine-containing peptides. According to the present invention, the nanoparticles allow highly selective isolation of target peptides in a simple and rapid manner. Therefore, the present invention can be applied to research on the treatment of diseases such as cancers.

### BACKGROUND ART

Recent biological research has focused on how to systemize, store and take advantage of a great deal of information acquired through long-term research. The amount of biological information available and produced in the current knowledge-based society is rapidly increasing. The rate of information production has also been increasing sharply since the base sequencing of the human genome was completed after launching of the human genome project.

The mass production of biological information has been led by DNA array, proteomics, combinatorial chemical libraries (CCLs), robotics-based high throughput screening (HTS), bioinformatics and other technologies. Sufficient amounts of biological and medical information have already been collected. It is estimated that humans have about one hundred thousand genes. The functions of most human genes except about ten thousand genes are still unknown. Functional characterization of unknown genes of non-human animal and plant species is an area of research in the future. Proteomics is a field of functional genetics to investigate the unknown functions of genes. Proteomic techniques are effectively utilized in investigating the functions of genes and proteins.

Proteomics can assist in the rapid provision of much information about the expression, modification and biochemical activity of disease-related genes and proteins in biology and other fields. The production and collection of useful information based on proteomics and effective utilization of the acquired information are of increasing interest in biological, medical and industrial fields. Thus, there is a need to introduce and rapidly establish mass information production methods in order to make progress in biological and medical research.

In recent years, many proteomic technologies have been proposed to overcome the limited dynamic ranges of current analytical platforms. In particular, techniques for enriching proteome subsets—such as glycopeptides, phosphopeptides, and cysteinyl peptides—according to their chemical or physical properties have been investigated to reduce the complexity of protein subsets present at low cellular abundance while increasing their concentrations and, thus, significantly increase the comprehensiveness and depth of the proteomic information.

Conventional techniques associated with the use of micron-sized beads for the selective isolation of peptides have limitations in terms of selectivity, efficiency and rapidity, which are lower than the levels required in high-sensitivity proteomic technologies. In addition, the conventional techniques have problems in that as the processing time increases, impurities deteriorate the selectivity of beads and cause loss of peptides during enrichment and washing.

Korean Unexamined Patent Publication No. 10-2004-0074807 discloses a method for producing magnetic nanoparticles for use in the isolation of a protein. According to the method, peptides or proteins present in low proportions cannot be selectively isolated from highly complex samples of proteins and must undergo additional isolation processing for proteomics experiments.

Further, Korean Patent No. 10-0762969 discloses a method for the isolation and purification of nucleic acids using functional silica-coated magnetic nanoparticles. Amine and alkyl groups are bound to the surfaces of the magnetic nanoparticles. The functional groups are bonded to phosphoric acid groups by an electrostatic attractive force, thus enabling the isolation of nucleic acids. However, the kind of nucleic acids or proteins isolated by the method is limited and the bonds formed by electrostatic interaction are very sensitive to processing conditions, such as solvents.

Further, Korean Unexamined Patent Publication No. 10-2007-0018501 proposes a method for the isolation and purification of DNA using silica-coated magnetic nanoparticles modified with amino groups. According to this method, however, target peptides cannot be selectively isolated and the captured peptides are again cleaved due to the hydrophobic interaction of alkyl groups during enrichment and washing.

None of the prior art methods suggest an alternative to the selective isolation of target peptides required in high-sensitivity proteomics.

### DISCLOSURE

[Technical Problem]

A first object of the present invention is to provide nanoparticles that can be used to highly selectively isolate target peptides from a mixture of peptides in a simple and rapid manner.

A second object of the present invention is to provide a method for producing the nanoparticles.

A third object of the present invention is to provide a method for the isolation of target peptides using the nanoparticles.

[Technical Solution]

In order to accomplish the first object of the present invention, nanoparticles are provided that comprise magnetic nanoparticles and thiol-specific functional groups as first functional groups bound to the surfaces of the magnetic nanoparticles to selectively capture cysteine-containing peptides.

In a preferred embodiment, the first functional group contains a 2-pyridyldisulfide group.

In a further embodiment, the magnetic nanoparticles may be coated with silica. In this embodiment, the first functional groups are bound to the silica layers.

Any group that is selective for target peptides may be used without any particular limitation as the first functional group. For example, the first functional group contains a thiol-specific 2-pyridyldisulfide group undergoing disulfide exchange with cysteine. As a result of the exchange reaction, the cysteine is covalently bonded to the nanoparticle. The covalent bond prevents the cysteine from being lost even during subsequent washing.

In another embodiment, the nanoparticle may further comprise second functional groups bound to the surfaces of the magnetic nanoparticles to prevent aggregation of the adjacent nanoparticles. In a preferred embodiment, the second functional group contains a phosphate group. The second functional group is negatively charged to generate a high negative zeta potential on the nanoparticle surface and stably maintain the dispersed state of the nanoparticle. Numerous functional groups may be used as the second functional groups. Preferably, the second functional group contains a phosphate group that is negatively charged and does not participate in the bonding with target peptides in the subsequent step.

So long as the magnetic nanoparticles can react with a magnetic material and are movable in the subsequent separation step, the kind of the magnetic nanoparticles is not particularly limited. For example, the magnetic nanoparticles may be selected from iron oxide, cobalt, nickel, and iron oxide doped with at least one material from manganese and zinc. In a preferred embodiment, the magnetic nanoparticles have a diameter of 25 to 35 nm. It is difficult to produce the magnetic nanoparticles having a diameter smaller than 25 nm. Meanwhile, the use of the magnetic particles larger than 35 nm increases the size of the functionalized nanoparticles, resulting in a reduction in the effective area of the functionalized nanoparticles for the capture of target peptides.

In order to accomplish the second object of the present invention, there is provided a method for producing nanoparticles for use in the isolation of peptides, the method comprising coating the surfaces of magnetic particles with silica, and binding thiol-specific functional groups as first functional groups to the silica layers to selectively capture cysteine-containing peptides.

In an embodiment, the coating step may include dispersing the magnetic particles and a dispersant in a solvent, adding ammonia (NH₃) and tetraethylorthosilicate (TEOS) to the dispersion of the magnetic particles to form silica layers on the surfaces of the magnetic particles, and separating the silica-coated magnetic particles from the solvent by precipitation.

In a preferred embodiment, the first functional group contains a 2-pyridyldisulfide group. The binding step may include dispersing the coated magnetic nanoparticles in a solvent, adding 3-aminopropyltriethoxysilane to the dispersion of the coated magnetic nanoparticles to bind functional groups containing 3-aminopropyl groups to the silica layers, and adding N-succinimidyl 3-(2-pyridyldithio)propionate to the dispersion of the nanoparticles functionalized with the 3-aminopropyl groups to bond 2-pyridyldisulfide groups to the 3-aminopropyl groups.

In a further embodiment, the method of the present invention may further comprise binding second functional groups to the silica layers to prevent aggregation of the adjacent nanoparticles. Preferably, the second functional group contains a phosphate group. The binding step may include dispersing the nanoparticles functionalized with the first functional groups in a solvent and adding 3-(trihydroxysilyl)propylmethylphosphonate to the dispersion.

In order to accomplish the third object of the present invention, there is provided a method for the isolation of peptides using magnetic nanoparticles, the method comprising mixing a peptide mixture containing target peptides with nanoparticles having first functional groups selectively capturing the target peptides, bonding the first functional groups of the nanoparticles to the target peptides, and separating the captured target peptides from the peptide mixture. The functional groups of the nanoparticles are covalently bonded to the target peptides. The separation step may be carried out by using a magnetic material to move the nanoparticles.

In an embodiment, the method of the present invention may further comprise isolating the target peptides from the nanoparticles after the separation step.

In a further embodiment, the first functional group may be a thiol-specific functional group. In another embodiment, the target peptides bonded to the first functional groups may be cysteinyl peptides. In a preferred embodiment, the thiol-specific functional group contains a 2-pyridyldisulfide group.

In another embodiment, the nanoparticles may further include second functional groups to prevent aggregation of the adjacent nanoparticles. In a preferred embodiment, the second functional group contains a phosphate group.

The nanoparticles include magnetic nanoparticles selected from iron oxide, cobalt, nickel, and iron oxide doped with at least one material from manganese and zinc. In a preferred embodiment, the surfaces of the magnetic nanoparticles may be coated with silica.

The method of the present invention can be applied to the isolation of target peptides from samples of proteins or serum. Proteins containing target peptides are generally reduced by pretreatment when it is intended to capture the target peptides using the nanoparticles of the present invention from the proteins. The nanoparticles of the present invention can also be applied to the isolation of naturally occurring cysteinyl peptides in a reduced state from proteins without any reduction pretreatment.

As can be seen from the following Examples section, the present inventors have succeeded in substantially perfectly isolating enolase proteins containing reduced cysteine. FIG. 5 is a schematic diagram showing a helical structure of an enolase protein used in the experiments of the present invention. Yeast enolase protein contains only one cysteine in its amino acid sequence. The cysteine forms a disulfide bond with the enolase molecules. Therefore, most enolase proteins exist in a dimeric form. Only some enolase proteins exist in a monomeric form because they contain cysteine in a reduced state. The nanoparticles of the present invention are characterized by their ability to selectively isolate and enrich small amounts of proteins containing cysteine in a reduced state.

The redox reactions of cysteine play an important role in metabolic control in living cells. The proportion of cysteine in a reduced state in a certain metabolic stage provides very useful information on the control of the metabolism and the incidence of related diseases. As described above, the present invention can provide a technique by which trace amounts of proteins containing reduced cysteine can be selectively separated from proteins containing oxidized cysteine in a protein mixture. Therefore, the nanoparticles of the present invention are very useful in investigating proteins, their metabolic roles and related diseases.

### DESCRIPTION OF DRAWINGS

In the figures:

FIG. 1 illustrates a method for producing nanoparticles for use in the isolation of peptides according to an embodiment of the present invention;

FIG. 2 shows transmission electron microscopy (TEM) images of nanoparticles (a) before and (b) after surface coating with TEOS in accordance with an embodiment of the present invention;

FIGS. 3a, 3b and 3c are chromatograms of standard peptides before isolation, peptides unbound to nanoparticles and a peptide captured by nanoparticles in Experimental Example 1, respectively;

FIG. 4 shows chromatograms of (a) an enolase protein before capturing, (b) peptides unbound to nanoparticles and (c) a peptide captured by nanoparticles in Experimental Example 2, and (d) peptides unbound to commercially available micron-sized agarose beads, (e) peptides captured by and released from commercially available micron-sized agarose beads after dispersion for one hour and (f) peptides captured by and released from commercially available micron-sized agarose beads after dispersion for one minute in Comparative Example 1;

FIG. 5 is a schematic diagram showing a helical structure of a typical enolase protein;

FIG. 6a is a chromatogram measured after enolase proteins were reduced in a monomeric form and isolated using nanoparticles in Experimental Example 3, and FIG. 6b is a chromatogram measured after reduced cysteine in a monomeric form was isolated from enolase proteins using nanoparticles in Experimental Example 3; and

FIG. 7 is a chromatogram of peptides isolated using nanoparticles in Experimental Example 4.

### MODE FOR INVENTION

Hereinafter, the present invention will be explained in more detail with reference to the following examples. However, these examples are given for the purpose of illustration and are not to be construed as limiting the scope of the invention.

### EXAMPLES

### Example 1: Production of Nanoparticles for Use in the Isolation of Peptides

### Example 1-1: Formation of Silica Layers

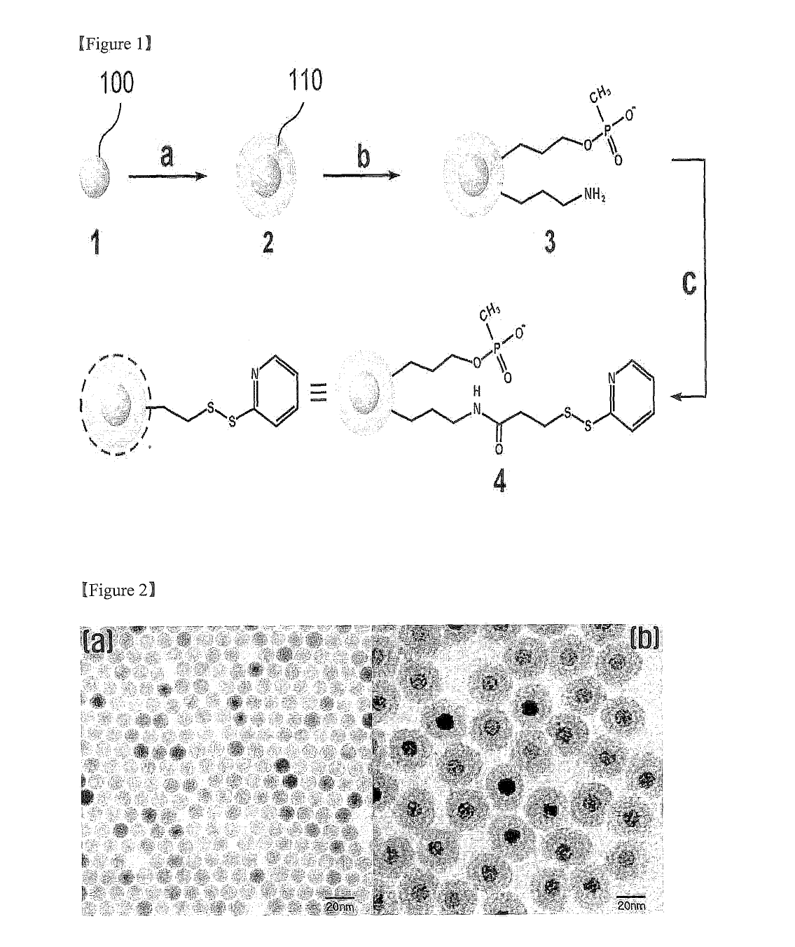

FIG. 1 illustrates a method for producing nanoparticles for peptide isolation according to an embodiment of the present invention. In step a, a silica layer 110 is formed on the surface of a Fe₃O₄ magnetic particle 100.

First, 90 mg of Fe₃O₄ (diameter=12 nm) and 2 mL of oleic acid (90%, Aldrich) were dispersed in 120 mL of cyclohexane as an apolar solvent by sonication at 50° C. for one hour to prepare Solution A. 24 g of a dispersant (Igepal CO-520, Aldrich) was dissolved in 300 mL of cyclohexane in a 1-L Erlenmeyer flask with magnetic stirring, and then Solution A and 300 mL of cyclohexane were added thereto. The mixture was stirred for 10 minutes. The reason for the use of the apolar solvent (cyclohexane) is as follows. The dispersant (Igepal) forms reverse micelles in the cyclohexane. The polar domains of the dispersant are present in the cores of the reverse micelles. It is preferred that the reverse micelles are small in size in order to uniformly form silica layers on the respective surfaces of the small nanosized magnetic particles. If the inner cores of the reverse micelles are large, two or more magnetic particles may be present in each micelle and share one silica layer in common after subsequent coating, unlike in FIG. 2 where a silica layer is coated on the surface of each of the magnetic particles.

Thereafter, a 28-40% aqueous solution of ammonia, which acts as a catalyst for the formation of silica layers on the nanoparticles, was added dropwise to the mixture through a syringe. After the mixture was stirred for 5 minutes, TEOS was added dropwise thereto to form silica layers (Si—O—Si) on the surfaces of the magnetic particles. Stirring was further continued at room temperature for 20 hours. 500 mL of methanol was added to precipitate the coated nanoparticles. The supernatant was discarded and the precipitate was collected by centrifugation at 3,500 rpm. The precipitate was purified through a series of sonication in hexane (3×90 mL), precipitation in chloroform (30 mL) and centrifugation (3,500 rpm). This purification procedure was repeated once more.

The resulting nanoparticles had a structure in which silica layers were formed on the surfaces of the core Fe₃O₄ particles. The dispersant (Igepal), which would impede the isolation of peptides in the subsequent step, adsorbed on the surfaces of the nanoparticles was removed from the coated nanoparticles.

FIG. 2 shows transmission electron microscopy (TEM) images of the nanoparticles (a) before and (b) after surface coating with the TEOS. The images of FIG. 2 show that the silica layers were uniformly coated on the surfaces of the respective spherical Fe₃O₄ particles.

### Example 1-2: Modification of the Coated Nanoparticles with 3-(trihydroxysilyl)propylmethylphosphonate (THPMP) and 3-aminopropyltriethoxysilane (APTS) (Step B of FIG. 1)

150 mg of the silica-coated nanoparticles in the form of powder obtained in Example 1-1 were mixed with 50 mL of methanol in a 250 mL round bottom flask, followed by sonication for one hour. 3-Aminopropyltriethoxysilane (2.75 μmol/mg, 75 μL, Aldrich) and 3-(trihydroxysilyl)propylmethylphosphonate (2.2 μmol/mg, 149.5 μL, Aldrich) were added to the flask and mechanically stirred using an impeller at 350 rpm. The mixture was refluxed at 80° C. for 7 hours, washed with methanol (3×25 mL), and dried to yield nanoparticles modified with the APTS and THPMP.

### Example 1-3: Surface Treatment of the Modified Nanoparticles with N-succinimidyl 3-(2-pyridyldithio)propionate (SPDP) (Step C of FIG. 1)

100 mg of the modified nanoparticles were dispersed in 9.5 mL of methanol by sonication for one hour. A solution of N-succinimidyl 3-(2-pyridyldithio)propionate (0.2 μmol/mg, 6.2 mg, Calbiochem) in 500 μL of DMSO was added to the dispersion, washed with methanol (3×25 mL), and dried to yield nanoparticles for use in the isolation of peptides. Functional groups containing thiol-reactive 2-pyridyldisulfide groups were covalently bound to the surfaces of the modified nanoparticles.

### Experimental Example 1: Separation of Four Standard Peptides Using the Functionalized Nanoparticles

A mixture of 1.0 nmol of somatostatin fragment 2-9 (GCKNFFWK), 0.64 nmol of bradykinin (RPPGFSPER), 1.28 nmol of angiotensin and 0.332 nmol of amyloid β fragment 17-28 (LVFFAEDVGSNK) as standard peptides was dissolved in degassed water to prevent the oxidation of cysteine. All the peptides were purchased from Sigma and were used in the next step without further purification.

Subsequently, the peptide mixture was mixed with 0.1 mg of the functionalized nanoparticles in 50 mM Tris and 10 mM EDTA (Tris-HCl buffer, pH 7.5) at room temperature for one minute. Then, the functionalized nanoparticles were separated from the mixture using a magnetic material (SmCo5 magnet) capable of reacting with the magnetic nanoparticles, and the supernatant was collected.

The separated nanoparticles were washed with Tris-HCl buffer (20 μL) to separate and collect the peptides remaining unbound to the nanoparticles. The collected supernatant and the washing solution were mixed together, and the washed nanoparticles were dispersed in a 0.1 M ammonium carbonate solution (20 μL). Then, 0.1M tris(carboxyethyl)phosphine (TCEP) was added to the dispersion to cleave the sulfur bonds between the cysteine and the nanoparticles and separate the cysteine captured by the nanoparticles. TCEP is merely illustrative, and it is to be understood that various methods known in the art may be used depending on the bonds between the nanoparticles and target peptides to be isolated.

Then, the mixture was washed to isolate the free peptide (‘Peptide A’) from the nanoparticles, and dried. The peptides (‘Peptides B’) were separated from the washing solution and the supernatant. Peptide A and Peptides B were analyzed by capillary LC/MS/MS.

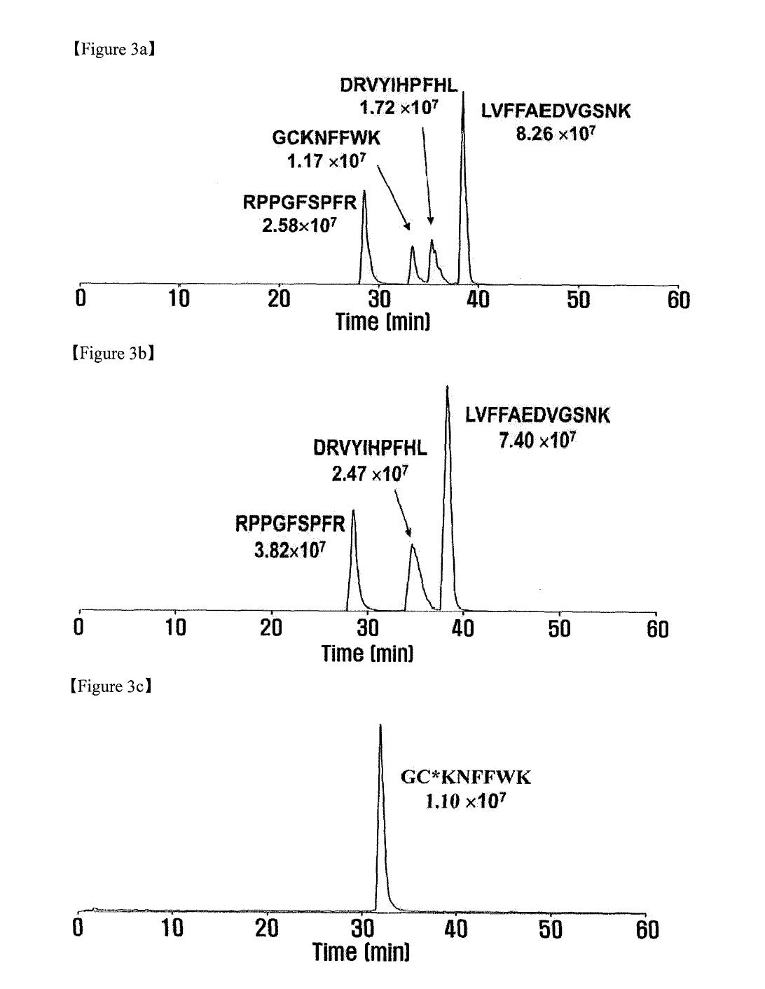

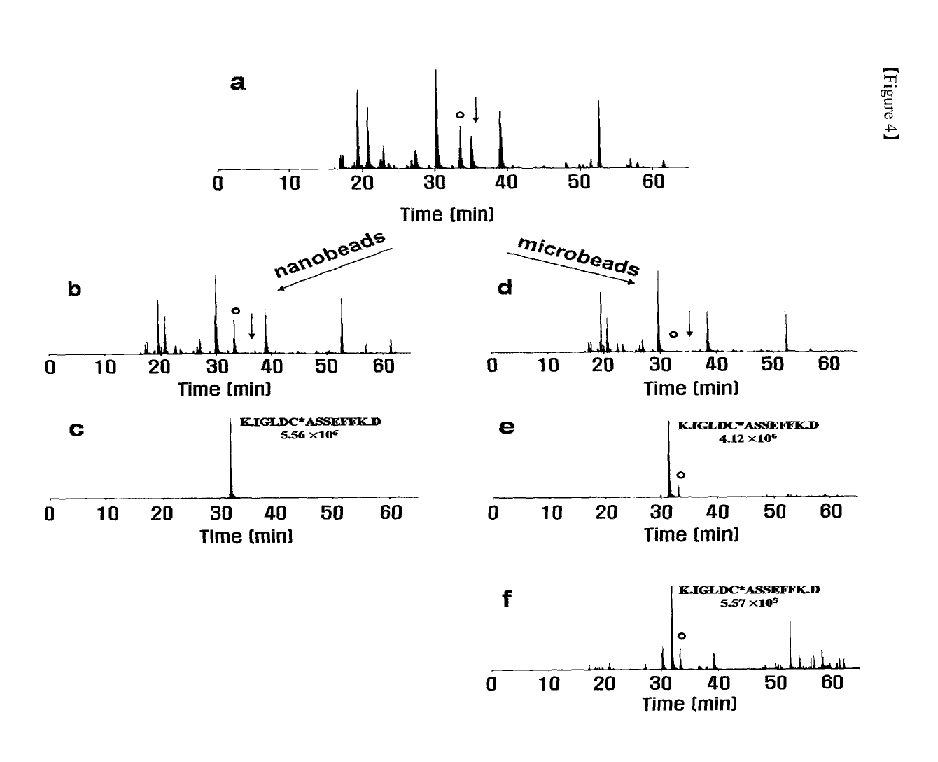

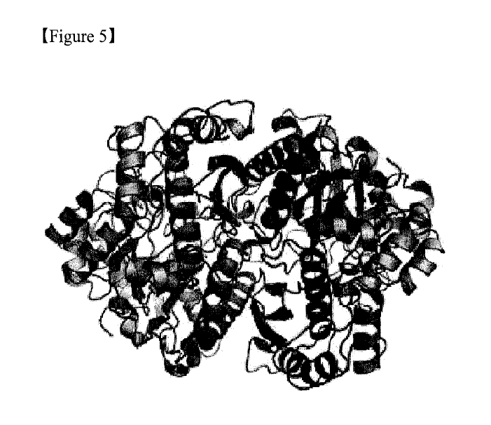

FIGS. 3a, 3b and 3c are chromatograms of the standard peptide mixture, Peptides B and Peptide A, respectively. Referring to FIG. 3a, cysteine (C) was present in the somatostatin fragment 2-9 and was absent in the other three peptides. Referring to FIG. 3b, cysteine (C) was absent in Peptides B unbound to the nanoparticles. Referring to FIG. 3c, Peptide A captured by the nanoparticles was the cysteine-containing somatostatin fragment 2-9.

From these experimental results, it can be confirmed that only the cysteinyl peptide could be selectively isolated due to the specific capture of the nanoparticles for cysteine.

### Experimental Example 2: Isolation of Enolase Proteins Using the Functionalized Nanoparticles (I)

1 μg of yeast enolase proteins were trypsinized in 25 mM ammonium carbonate buffer. The tryptic peptides were reduced with 0.1 M TCEP at 25° C. for 30 minutes.

The pretreated peptides were captured by the nanoparticles in the same manner as in Experimental Example 1. The peptides unbound to the nanoparticles and the peptide captured by the nanoparticles were separated, collected and analyzed by capillary LC/MS/MS.

FIG. 4 shows chromatograms of (a) the peptides before the capturing, (b) the peptides unbound to the nanoparticles and (c) the peptide captured by the nanoparticles.

Referring to FIG. 4, cysteine (C) was present in the enolase peptide captured by and released from the nanoparticles and was absent in the peptides unbound to the nanoparticles. These results demonstrate that the nanoparticles produced in Example 1 are highly selective for cysteine and are effective in isolating cysteinyl peptides.

### Comparative Example 1: Isolation of Enolase Proteins Using Thiopropyl Sepharose 6B

The procedure of Experimental Example 2 was repeated except that thiopropyl sepharose 6B was used instead of the nanoparticles. The thiopropyl sepharose 6B is commonly used for cysteine isolation. The thiopropyl sepharose 6B were hydrated in water for 15 minutes before experiments. The results are shown in FIG. 4.

FIG. 4 shows chromatograms of (d) the enolase peptides captured by and unbound to the thiopropyl sepharose 6B, (e) the peptides released from the thiopropyl sepharose 6B after dispersion in the enolase peptide solution for one hour, and (f) the peptides released from the thiopropyl sepharose 6B after dispersion in the enolase peptide solution for one minute.

The peak intensity of the base peak chromatogram (4f) of the peptides released from the thiopropyl sepharose 6B after dispersion in the enolase peptide solution for the same time (i.e. 1 min.) was less than one tenth of that of the base peak chromatogram (4c) of the cysteinyl peptide captured by the nanoparticles. In addition, many peaks corresponding to nonspecifically interacting peptides other than the cysteinyl peptide were observed in the chromatogram (4f).

The base peak chromatogram (4e) of the peptides released from the thiopropyl sepharose 6B after dispersion in the enolase peptide solution for one hour had a peak intensity similar to the base peak chromatogram (4c) of the cysteinyl peptide captured by the nanoparticles, but many peaks corresponding to nonspecifically interacting peptides other than the cysteinyl peptide were observed in the chromatogram (4e).

The results in Comparative Example 2 and FIG. 4 demonstrate that the nanoparticles produced in Example 1 showed much better results in terms of capturing efficiency and selectivity over the conventional micron-sized thiopropyl sepharose 6B beads.

### Experimental Example 3: Isolation of Enolase Proteins Using the Functionalized Nanoparticles (II)

1 μg of a sample of yeast enolase proteins was dissolved in 50 mM Tris and 10 mM EDTA (Tris-HCl buffer, pH 7.5). Most enolase proteins are oxidized and contain cysteinyl disulfide bonds. Only some enolase proteins contain cysteine in a reduced state. In this experiment, a portion of the enolase proteins containing cysteine in a reduced state was selectively isolated and purified. To this end, the proteins underwent no reduction to prevent the proteins from being bound to the nanoparticles before reaction, unlike in Experimental Example 2.

Meanwhile, 1 μg of another sample of yeast enolase proteins was reduced by pretreatment with 0.1 M TCEP at 25° C. for 30 minutes to convert the dimeric form of the enolase proteins to a monomeric form.

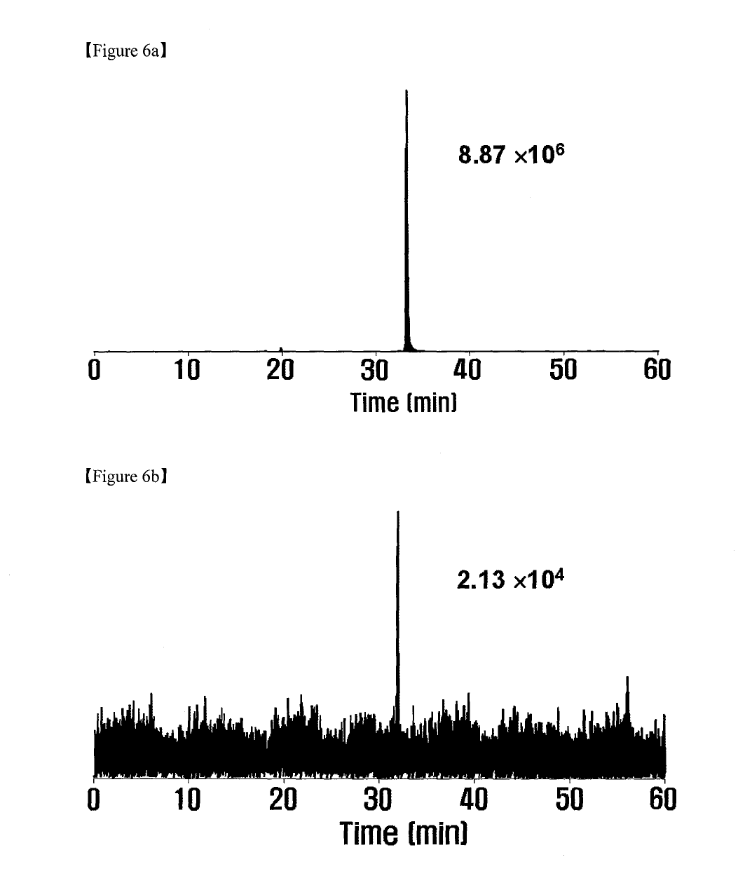

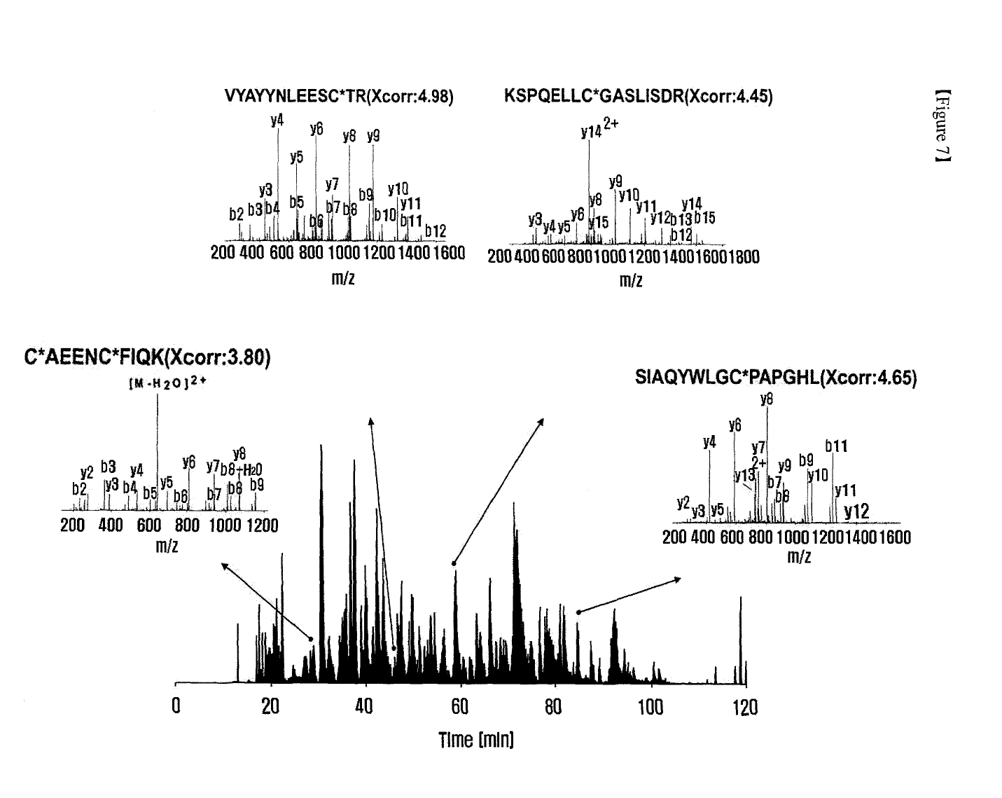

The two protein solutions were mixed with 0.1 mg of the nanoparticles produced in Example 1 at room temperature for one hour. The subsequent procedure was repeated in the same manner as in Experimental Example 2. The obtained results are shown in FIGS. 6a and 6a. FIG. 6a is a chromatogram showing the peptide ingredients of the enolase proteins that were reduced, captured by and released from the nanoparticles. The peak indicates the total amount of the cysteinyl peptides because all the enolase proteins were in a reduced state.

FIG. 6b is a chromatogram showing the peptide ingredients of the enolase proteins that were not reduced, captured by and released from the nanoparticles. The two chromatograms reveal the ratio of the amount of the enolase proteins in a monomer state containing cysteine in a reduced state to the amount of the enolase proteins in a dimeric form, most of which contain cysteine in an oxidized state.

### Experimental Example 4: Isolation of Peptides from Human Serum Using the Functionalized Nanoparticles

In this experiment, the nanoparticles produced in Example 1 were applied to the selective isolation of cysteinyl peptides from samples of a human serum. The isolated peptides were analyzed by capillary LC/MS/MS. The analytical results are shown in FIG. 7.

FIG. 7 is a chromatogram of a complex mixture of a total of 456 peptides. 450 (98.6%) of the peptides were identified as cysteinyl peptides. Interestingly, the signals of any non-cysteine containing peptides were present at negligible intensities (i.e., less than 0.1% of the base peak intensity).

The above results indicate that the nanoparticles of the present invention are effectively covalently bonded to cysteine to achieve high selectivity for cysteine and low loss after bonding. In addition, the nanoparticles of the present invention are not chemically bound to peptides containing no cysteine, confirming that the nanoparticles are very effective in the isolation of cysteinyl peptides.

### INDUSTRIAL APPLICABILITY

As is apparent from the foregoing, the present invention provides nanoparticles in which first functional groups capable of selectively reacting with target peptides are bound to the surfaces of magnetic nanoparticles. The nanoparticles of the present invention enable selective isolation of desired peptides from peptide mixtures and proteins in a simple and rapid manner. Therefore, the nanoparticles of the present invention can find applications in various research fields, including therapeutic agents for protein metabolic diseases.

1. Nanoparticles comprising magnetic nanoparticles and thiol-specific functional groups as first functional groups bound to the surfaces of the magnetic nanoparticles to selectively capture cysteine-containing peptides.

2. The nanoparticles of claim 1, wherein the first functional group contains a 2-pyridyldisulfide group.

3. The nanoparticles of claim 1, wherein the magnetic nanoparticles are coated with silica and the first functional groups are bound to the silica layers.

4. The nanoparticles of claim 1, further comprising second functional groups bound to the surfaces of the magnetic nanoparticles to prevent aggregation of the adjacent nanoparticles.

5. The nanoparticles of claim 4, wherein the second functional group contains a phosphate group.

6. The nanoparticles of claim 1, wherein the magnetic nanoparticles are selected from iron oxide, cobalt, nickel, and iron oxide doped with at least one material from manganese and zinc.

7. The nanoparticles of claim 1, wherein the magnetic nanoparticles have a diameter of 25 to 35 nm.

8. A method for producing nanoparticles for use in the isolation of peptides, the method comprising
coating the surfaces of magnetic particles with silica, and
binding thiol-specific functional groups as first functional groups to the silica layers to selectively capture cysteine-containing peptides.

9. The method of claim 8, wherein the coating step includes dispersing the magnetic particles and a dispersant in a solvent, adding ammonia (NH₃) and tetraethylorthosilicate (TEOS) to the dispersion of the magnetic particles to form silica layers on the surfaces of the magnetic particles, and separating the silica-coated magnetic particles from the solvent by precipitation.

10. The method of claim 8, wherein the first functional group contains a 2-pyridyldisulfide group.

11. The method of claim 10, wherein the binding step includes dispersing the coated magnetic nanoparticles in a solvent, adding 3-aminopropyltriethoxysilane to the dispersion of the coated magnetic nanoparticles to bind functional groups containing 3-aminopropyl groups to the silica layers, and adding N-succinimidyl 3-(2-pyridyldithio)propionate to the dispersion of the nanoparticles functionalized with the 3-aminopropyl groups to bond 2-pyridyldisulfide groups to the 3-aminopropyl groups.

12. The method of claim 8, further comprising binding second functional groups to the silica layers to prevent aggregation of the adjacent nanoparticles.

13. The method of claim 12, wherein the second functional group contains a phosphate group.

14. The method of claim 13, wherein the binding step includes dispersing the nanoparticles functionalized with the first functional groups in a solvent and adding 3-(trihydroxysilyl)propylmethylphosphonate to the dispersion.

15. A method for the isolation of peptides using magnetic nanoparticles, the method comprising
mixing a peptide mixture containing target peptides with nanoparticles having first functional groups selectively capturing the target peptides,
bonding the first functional groups of the nanoparticles to the target peptides, and
separating the captured target peptides from the peptide mixture.

16. The method of claim 15, wherein the functional groups of the nanoparticles are covalently bonded to the target peptides.

17. The method of claim 15, wherein the separation step is carried out by using a magnetic material to move the nanoparticles.

18. The method of claim 15, further comprising isolating the target peptides from the nanoparticles after the separation step.

19. The method of claim 15, wherein the first functional group is a thiol-specific functional group and the target peptide is a cysteinyl peptide.

20. The method of claim 19, wherein the thiol-specific functional group contains a 2-pyridyldisulfide group.

21. The method of claim 15, wherein the nanoparticles further include second functional groups to prevent aggregation of the adjacent nanoparticles.

22. The method of claim 21, wherein the second functional group contains a phosphate group.

23. The method of claim 15, wherein the nanoparticles include magnetic nanoparticles selected from iron oxide, cobalt, nickel, and iron oxide doped with at least one material from manganese and zinc.

24. The method of claim 15, wherein the surfaces of the magnetic nanoparticles are coated with silica.

25. The method of claim 15, wherein the peptide mixture is a sample of proteins or serum.

26. The method of claim 25, wherein the target peptides captured by the nanoparticles are peptides or proteins containing cysteine reduced by pretreatment or naturally occurring cysteine in a reduced state.

27. The method of claim 26, further comprising measuring the proportion of the proteins containing naturally occurring cysteine in a reduced state captured by the nanoparticles in all cysteine-containing proteins.
